(19) United States
(12) Patent Application Publication
(10) Pub. No.: US 2015/0224020 A1
(43) Pub. Date: Aug. 13, 2015

(54) HOME-USE COSMETIC TREATMENT DEVICE

(71) Applicant: Syneron Beauty LTD, Yoqneam Illit (IL)

(72) Inventors: Lion Flyash, Nazareth Illit (IL); Fabian Tanenbaum, Tenafl (IL)

(21) Appl. No.: 14/417,556

(22) PCT Filed: Jul. 22, 2013
(86) PCT No.: PCT/IL13/00061

§ 371 Date: Jan. 26, 2015

Related U.S. Application Data
(60) Provisional application No. 61/675,973, filed on Jul. 26, 2012.

(51) Int. Cl. A61H 23/02; A61B 5/01; A61B 5/053; A61B 5/00

(52) U.S. Cl. CPC A61H 23/02; A61B 5/0059; A61B 5/01; A61B 5/0531

(57) ABSTRACT

An in home-use cosmetic body contouring device, including a head including an oscillating carrier, a convenient to hold handle integrally attached to the head and at least one curvilinear RF electrode and optionally at least one light energy emitting element.

### TECHNICAL FIELD

The method and apparatus are related to the field of personal cosmetic procedures and in particular to cellulite removal and body contouring procedures.

### BACKGROUND

External appearance is important practically to every person. Two of the most common skin changes affecting the contour of the body are cellulite, which occurs mostly in post-pubertal females and adipose tissue increase. Adipose tissue or body fat is located in the abdominal area although fat deposits in other body segments.

Cellulite presents as a change in the appearance of the surface of the skin evident by skin dimpling and nodularity that occurs mainly in women in the pelvic region, lower limbs and abdomen. Cellulite is caused by bulging of subcutaneous fat within fibrous connective tissue, leading to a padded or orange peel-like appearance.

In recent years, methods and apparatuses have been developed for different cosmetic or aesthetic treatments and especially for the treatment of cellulite.

Cosmetic body shaping treatments involving adipose tissue , also termed body contouring treatments, commonly involve employing complex devices and numerous methods of treatments to reduce body fat. These devices and treatments include application of various forms of heating energy, mechanical energy and similar.

Most, if not all of these treatments are performed by professionals in dedicated cosmetic clinics. Over time, a demand has increased for home care devices to allow users to self-provide skin care, such as body contouring in the privacy of their own homes and at a time most convenient for them.

Meeting this demand requires the development of mobile, easy to use and safe skin care cosmetic devices.

### SUMMARY

The present disclosure seeks to provide an in home-use cosmetic light-weight and safe body contouring and cellulite treatment device to allow users to self-provide skin care, such as body contouring and cellulite cosmetic treatment in the privacy of their own homes and at a time most convenient for them.

There is therefore provided a device having a head and a convenient to hold handle. The head could include a base integrally attached to the handle and have a bore defined by a wall having a rim and operative to rotatingly accommodate a rotatable carrier.

In accordance with an example the carrier could be depressed inside the a sink portion of the bore defined by the wall rim extending above the surface of the carrier so that when the head is applied to the tissue surface, only a limited area of the tissue surface defined by a radius of the wall rim could be urged into the sink portion and is allowed to come in contact with the carrier surface and electrodes.

In accordance with another example the mechanical energy in the form of vibration energy could also be applied only to the surface of the tissue urged into the sink portion to be cosmetically treated.

In accordance with another example, the carrier could also include a first surface to which one or more rigid or semi-rigid curvilinear RF electrodes could be attached. Additionally and optionally, one or more light emitting elements could also be attached to the first surface, in between or peripherally to the electrodes or to a rim of a vertical wall defining the bore in the base.

In accordance with yet another example, the carrier surface could also include one or more sensors communicating with a controller housed in the handle or base. The sensors could be one or more sensors selected from a group of sensors including temperature sensor, pressure sensor, optical sensor, skin impedance sensor and/or movement or positioning sensors.

In accordance with still another example, the device head or handle could also include one or more sources of power or a connector to an external source of power, activating buttons, a controller, electronic circuitry, one or more motors attached to and operative to rotate the carrier.

In accordance with still another example, the carrier could also include a vibrating electrical motor attached to a surface of the carrier to exert a massaging effect on a tissue surface to which it is applied.

### BRIEF DESCRIPTION OF THE DRAWINGS

The present invention will be understood and appreciated more fully from the following detailed description, taken in conjunction with the drawings in which:

FIGS. 1 is a plan view simplified illustration of a home-use cosmetic body contouring and cellulite treatment device in accordance with an example;

FIGS. 2A, 2B and 2C are side view simplified illustrations of a home-use cosmetic body contouring and cellulite treatment device in accordance with an example;

FIGS. 3A and 3B are cross-section view simplified illustrations of heads of home-use cosmetic body contouring and cellulite treatment device in accordance with two examples;

FIG. 4A, 4B and 4C are cross-section simplified illustrations of a massaging effect of a carrier of a cosmetic device head in accordance with several other examples;

FIGS. 5A and 5B are cross-section view simplified illustrations of heads of home-use cosmetic body contouring and cellulite treatment device in accordance with two other examples;

FIGS. 6A, 6B, 6C and 6D are plan view simplified illustrations of light emitting elements of a home-use cosmetic body contouring and cellulite treatment device oscillating head in accordance with an example;

FIG. 7 is a plan view simplified illustration of a home-use cosmetic body contouring and cellulite treatment device in accordance with an example;

FIGS. 8A and 8B are plan view simplified illustrations of a home-use cosmetic body contouring and cellulite treatment device in accordance with an example; and

FIGS. 9A, 9B and 9C are plan view simplified illustrations of a massaging effect of oscillating curvilinear electrodes on skin in accordance with an example.

### DETAILED DESCRIPTION

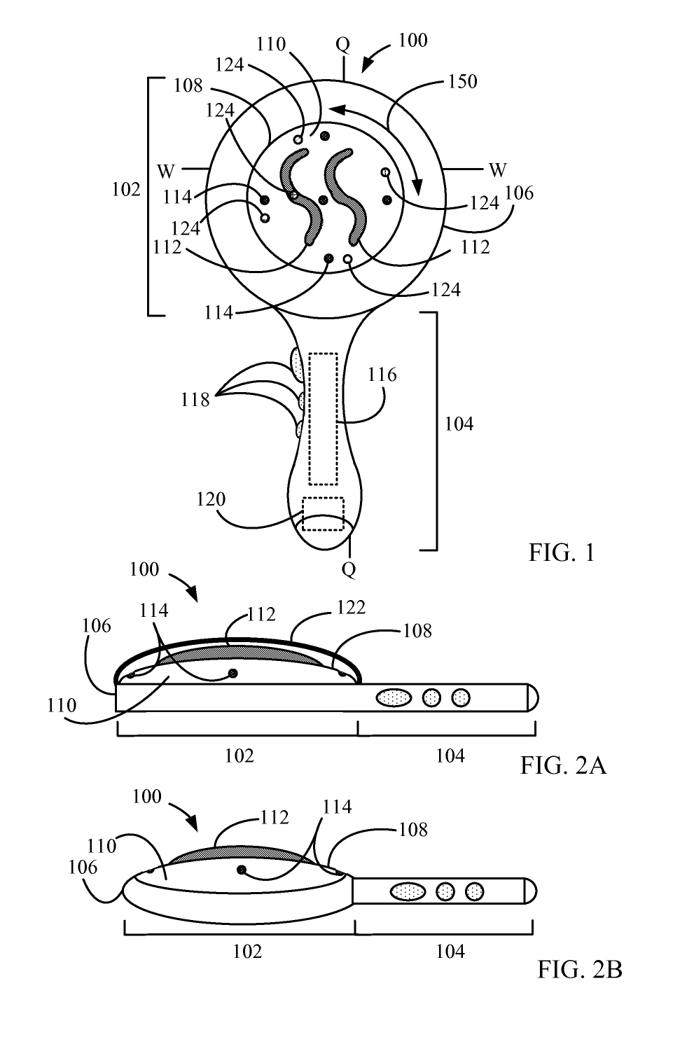

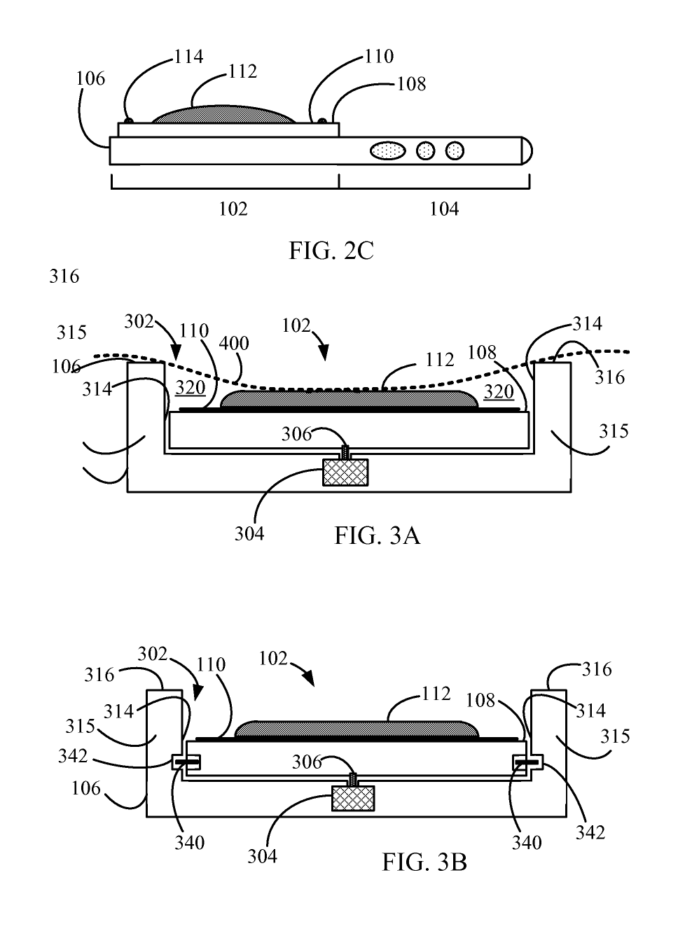

Referring now to FIGS. 1, 2A, 2B and 2C, which are a plan view and side-view simplified illustrations of a in home-use cosmetic body contouring and cellulite treatment device in accordance with an example. A light-weight body contouring and cellulite treatment device 100 includes a head 102 and a convenient to hold handle 104. Head 102 could be discoid in shape or have any other suitable shape and a generally flat cross-section. Alternatively and optionally head 102 could have an oval (FIG. 2A) or semi-oval (FIG. 2B) cross-section and include a base 106 integrally attached to handle 104 and having a centrally located bore 302 (FIG. 3) operative to rotatingly accommodate a rotatable carrier 108. Base 106 could be hollow so that to accommodate various electrical components such as electrical circuitry and wiring.

Rotatable carrier 108 could be operative to rotate up to 180 degrees or oscillate in a clockwise or counter clockwise direction within base 106. Most commonly carrier 108 could be operative to oscillate as indicated by arrow 150. Optionally, carrier 108 could be a passive or active “floating” carrier operative to apply uniform pressure to the surface of a body, adjust to the contour of the body and/or apply mechanical energy in the form of vibration energy to the surface of the body to be cosmetically treated as will be explained in greater detail below. A surface 110 of rotatable carrier 108 could be curved (FIGS. 2A and 2B or flat (FIG. 2C) and include one or more rigid or semi-rigid curvilinear (e.g., S-shaped) RF electrode 112. Additionally and optionally, surface 110 could also include one or more light emitting elements 114 selected from a group of light energy emitting elements including a Light Emitting Diode (LED), laser source, intense pulse light (IPL), infrared (IR) energy emitter or similar.

Electrodes 112 could have a rounded cross-section and protrude between 1 and 10 mm from surface 110 as shown in FIGS. 2A and 2B. The rounded cross-section prevents a common phenomenon in which electrical charge and current concentrate along sharp corners and edges of an electrode causing discomfort to the user.

Additionally or alternatively and optionally, head 102 could include one or more sensors 124 communicating with controller 120 in handle 104 or base 106. Sensors 124 could be located on surface 110 of carrier 108 and/or on one or more electrodes 112. Sensors 124 could be one or more sensors selected from a group of sensors including temperature sensor, thermopile infrared sensors, pressure sensor, optical sensor, and/or movement/or positioning sensors. Skin impedance could be sensed through electrodes 112.

Sensors 124 could be arranged along rim 316 (FIGS. 3A and 3B) or distributed on surface 110 around and/or between RF electrodes 112.

Additionally and optionally, head 102 could also include a rigid head 102 cover 122 (FIG. 2A) removably attached to base 106.

Handle 104 could include one or more sources of power 116, such as a rechargeable battery or an external source of AC or DC power, activating buttons 118 activating cosmetic body contouring and cellulite treatment device 100, a controller 120 to activate and control RF electrode 112 and light emitting elements 114 and electronic circuitry (not shown) for activating and controlling head 102 electrical components such as electrodes 112, one or more motors 304 (FIG. 3A) as will be explained in greater detail below.

Alternatively and optionally, source of power 116, activating buttons 118, controller 120, motor 304 and electronic circuitry (not shown) could be housed in head 102.

Referring now to FIGS. 3A and 3B, which are cross-section view simplified illustrations, taken along an axis Q-Q (FIG. 1), of heads of home-use cosmetic body contouring and cellulite treatment device in accordance with several examples. As shown in FIG. 3A, base 106 could include a centrally located bore 302, defined by a floor 308 and a ring-like vertical wall 315 including a rim 316 and operative to accommodate rotatable carrier 108. In some examples, rim 316 could have rounded edges. Carrier 108 could be attached to an oscillating electrical motor 304 via a rigid, telescopic or flexible rotatable drive 306. Motor 304 could be selected from a group of motors including brushless motors, ac or dc motors, stepper motors or piezoelectric motors and when incorporated in handle 104 could drive carrier 108 via a mechanical transmission.

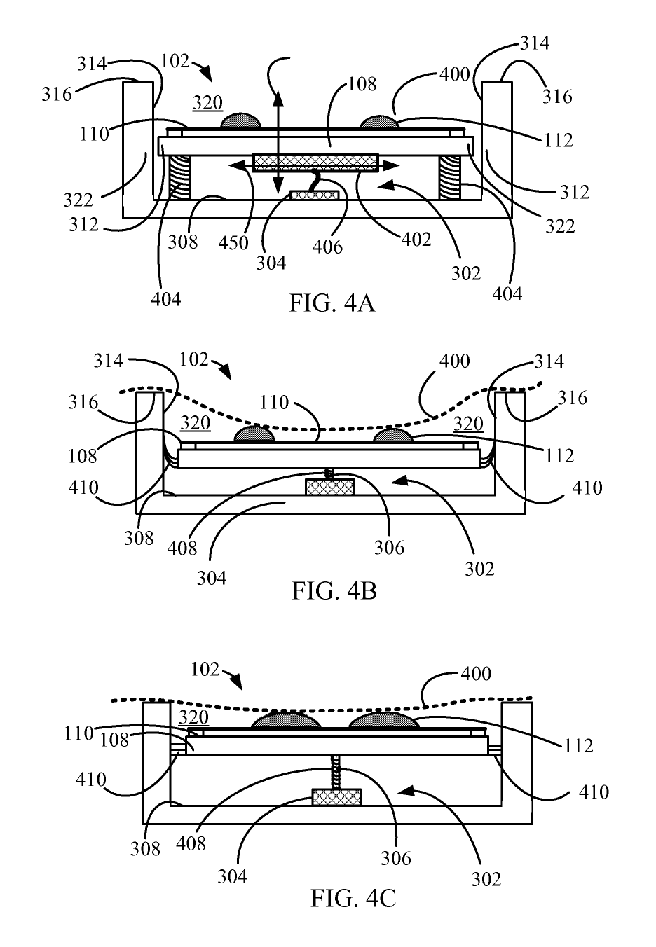

Carrier 108 could be depressed inside bore 302 so that wall 315 rim 316 can extend 1-10 mm above surface 110 of carrier 108. This configuration could create a sink portion 320 of bore 302 defined by wall 315, an imaginary plane at the level of rim 316 and normal to wall 315 and carrier 108 surface 110 so that and as shown in FIG. 3A, when head 102 is applied to tissue surface 400 (FIG. 4), only a limited area of tissue surface 400 (indicated in FIG. 3A as a broken line) defined by a radius of wall 315 rim 316 is urged into sink portion 320 and is allowed to come in contact with carrier 108 surface 110 and electrodes 112. Wall 315 rim 316 that extends 1-10 mm above surface 110 of carrier 108 absorbs or receives most of the pressure applied by the tissue surface 400 (indicated in FIG. 3A as a broken line). Because of this, the segment of the tissue urged into sink portion 320 and that is in contact with carrier 108 surface 110 and electrodes 112 does not apply excessive pressure to axis 306 of motor 304. Although in some examples axis 306 could be a telescopic axis.

Alternatively and optionally and as shown in FIG. 3B, carrier 108 could also include horizontally rotating wheels 340, vertically slidable and/or rotatable within a groove 342 in wall 315 centrally maintaining carrier 108 inside bore 302.

Reference is now made to FIGS. 4A, 4B, and 4C, which are cross-section view simplified illustrations, taken along an axis W-W (FIG. 1), depicting massaging effect of a carrier in according with two examples.

As shown in FIG. 4A, massaging effect of a carrier 108 could be generated for example, by an eccentric rotating mass. A vibrating coin-type or other electrical motor 402 attached to a surface of carrier 108 opposite to surface 110 could be coupled to rotate the mass or vibrate directly the carrier 108. Carrier 108 could rest on one or more bias 404 operative to vertically contract and expand as well as be partially deformed under shearing forces so that to allow oscillation of carrier 108.

Motor 402 could induce a vertical vibration motion , perpendicular to surface 110 as depicted by double-headed arrow 450, or parallel to surface 110 as depicted by double-headed arrow 470.

Additionally and optionally, carrier 108 could be brought to oscillate by a motor 304 connected to carrier 108 via a flexible drive 406.

FIGS. 4B and 4C depict another example, in which an oscillating electrical motor 304 is attached to carrier 108 in an arrangement similar to that depicted in FIG. 3A, driving carrier 108 via a rotatable telescopic drive 306 housed within a bias 408.

Additionally, carrier 108 could also be supported along its periphery by one or more pliable lines 410 operative to allow limited oscillation of carrier 108.

In FIG. 4B, pliable lines 410 are shown to be in a resting loose position and carrier 108 is pulled towards motor 304 by bias 408, accommodating tissue surface 400 urged into sink portion 320 of bore 302 when head 102 is applied to tissue surface 400. When oscillating, and as shown in FIG. 4C, partial rotation of carrier 108 brings about increased tension in pliable lines 410 pulling carrier 108 vertically and away from motor 304 against tissue surface 400, forcing tissue surface 400 out of sink portion 320 of bore 302 and loading bias 408. Rotation in the opposite direction brings about loosening of pliable lines 410, allowing bias 408 once again to vertically pull carrier 108 towards motor 304 as depicted in FIG. 4B, once again accommodating tissue surface 400 urged into sink portion 320 of bore 302 when head 102 is applied to tissue surface 400. This mechanism mechanically exerts a massaging effect on and perpendicular to a limited portion of tissue surface 400 accommodated in sink 320 during oscillation of carrier 108.

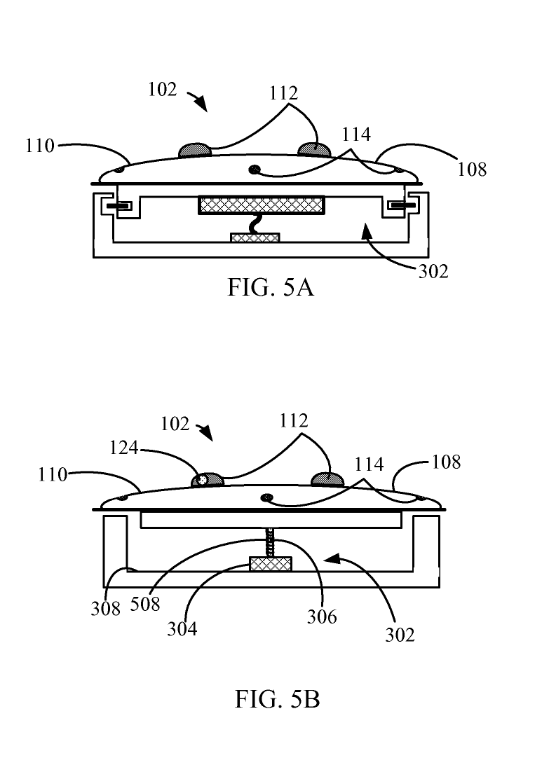

In other examples depicted in FIGS. 5A and 5B, which are cross-section view simplified illustrations, taken along an axis W-W (FIG. 1), of heads of home-use cosmetic body contouring and cellulite treatment device in accordance with two examples. Carrier 108 surface 110 could be curved and extend beyond rim 316 of base 106. FIG. 5A depicts a configuration similar to that shown in FIG. 4A whereas FIG. 5B depicts a configuration similar to that shown in FIG. 3A. Carrier 108 surface 110 could be rigid or be pliable to the touch increasing comfort of application of head 102 to body contours. In cases in which surface 110 is pliable, electrodes 112 could also be semi-rigid adding to the comfort of application of head 102 to body contours.

In FIG. 5B, a sensor 124, such as a temperature or pressure sensor is shown to be located on RF electrode/s 112. Optionally, sensor 124 could be an impedance sensor paired with another sensor (not shown) on the other electrode 112 attached to surface 112. In this configuration, RF electrodes 112 could apply pulsed RF energy to tissue surface 400 (FIG. 4) and measure tissue impedance between RF pulses.

Additionally and optionally, carrier 108 could also include one or more temperature infrared sensors as described above.

Reference is now to FIGS. 6A, 6B, 6C and 6D, which are plan view simplified illustrations of light emitting elements of a home-use cosmetic body contouring and cellulite treatment device oscillating head 102 (FIG. 1) in accordance with an example. As shown in FIG. 6A, light emitting elements 114 could be arranged concentrically around RF electrodes 112 on surface 110 of carrier 108 and oscillate therewith. Alternatively and optionally and as depicted in FIG. 6B, light emitting elements 114 could be arranged in a fixed concentric pattern around RF electrodes 112 along rim 316 of base 106. FIG. 6C illustrates an arrangement of light emitting elements 114 on a surface 110 of carrier 108 such as that illustrated in FIGS. 5A and 5B. In this example, electrodes 112 are non S-shaped curvilinear electrodes. As shown in FIG. 6D, light emitting elements 114 could be distributed over surface 110 of carrier 108 as well as in-between electrodes 112.

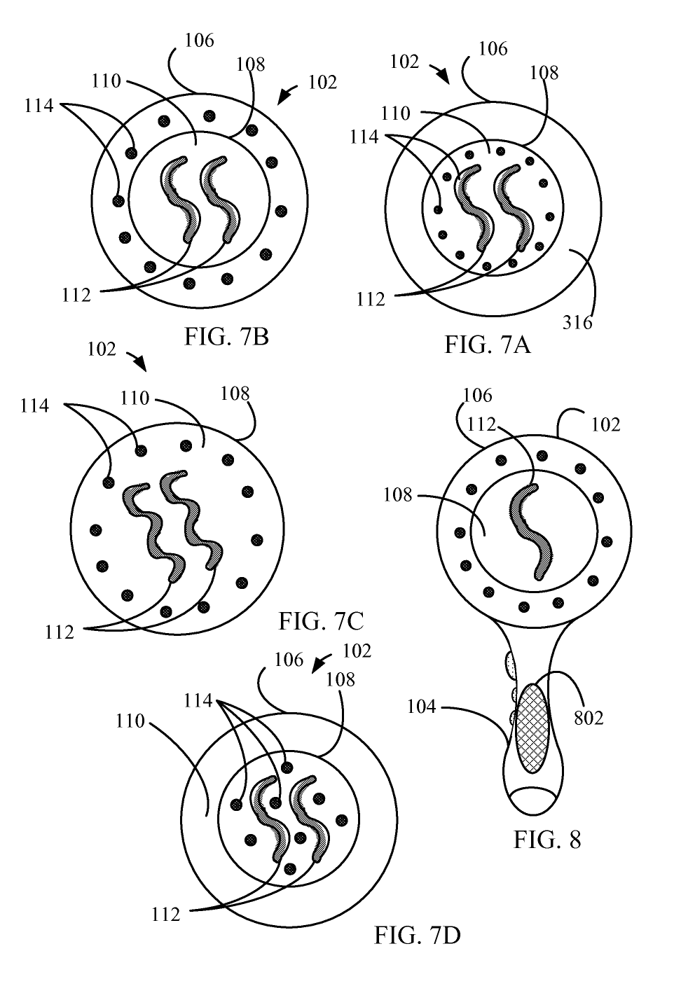

FIG. 7, which is a plan view simplified illustration of a home-use cosmetic body contouring and cellulite treatment device in accordance with an example, illustrates a single RF electrode device. In this configuration, a return electrode 702 could be embedded or attached to handle 104 so that to contact the body of a user when handle 104 is gripped by the hand of the user.

FIGS. 8A and 8B, which are plan view simplified illustrations of a home-use cosmetic body contouring and cellulite treatment device in accordance with an example, demonstrate the advantage of curvilinear (e.g., S-shaped) electrodes over commonly used linear electrodes. In an example, employing S-shaped electrodes 112 have a rounded cross-section as well as rounded tips 802 to prevent a common phenomenon in which electrical charge and current concentrate along sharp corners and edges of an electrode causing discomfort to the user.

The curvilinear shape of electrodes 112 facilitates the user to apply to a given area of body surface a dose of cosmetic treatment energy that could not be applied by a linear electrode 812. In FIGS. 8A and 8B the lengths of electrodes 112 and 812 are drawn to scale and are the same.

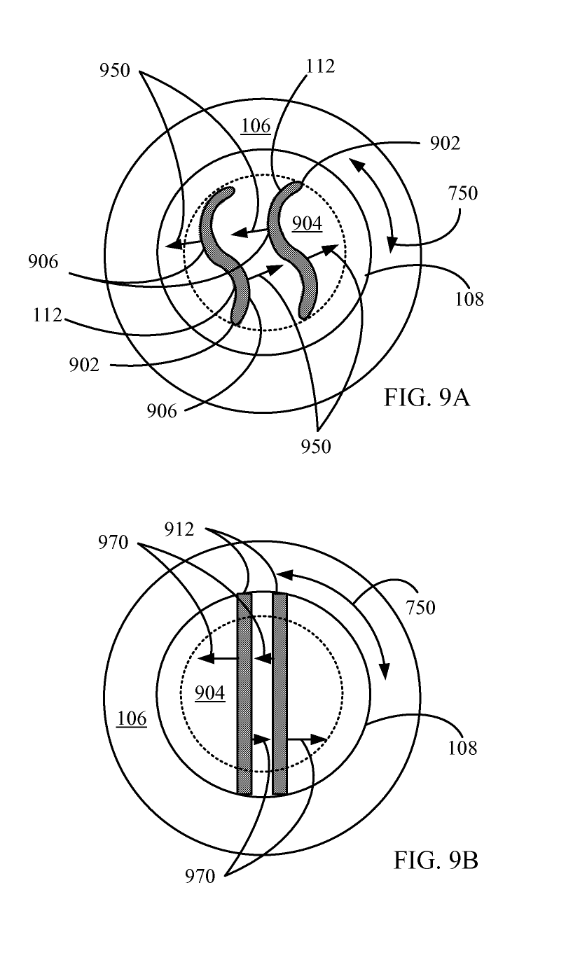

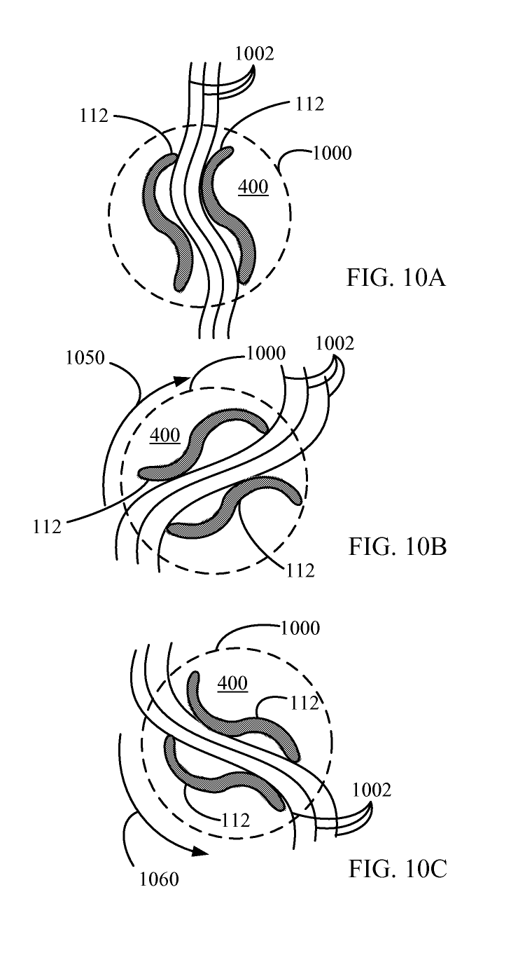

An area 804 treated by oscillating electrodes 112/812 is depicted by a broken line circle. It will be appreciated by a person skilled in the art that wherein in FIG. 9A electrodes 112 are capable of applying energy to area 804 along the full extent of their length, electrodes 812 (FIG. 9B) are capable of applying energy to area 804 only along a portion of their length thus making electrodes 112 more efficient, compact and suitable for home-use devices. Moreover, and as will be explained in greater detail below, when oscillating, S-shaped electrodes 112 include rounded fronts 806 that are much more comfortable to a user when moving against surface 400 (FIG. 4) of a body, as depicted by arrows 850, in a direction indicated by arrow 750 as compared to straight fronts of electrodes 812 as depicted by arrows 870.

Referring now to FIGS. 9A, 9B and 9C, which are plan view simplified illustrations of a massaging effect of oscillating curvilinear electrodes on skin in accordance with an example. In FIGS. 9A-9C, for purposes of illustration only, a portion of tissue surface 400 urged into sink portion 320 (FIGS. 3A and 4B) is depicted by a broken line circle designated reference numeral 900. Also for purposes of illustration, only electrodes 112 are depicted in the figures. As shown in FIG. 9A, body contouring and cellulite treatment device 100 has been applied to a surface 400 of a body urging tissue between electrodes 112 and creating tissue folds 902. Oscillation of surface 110 (FIG. 1) brings about partial rotation of electrodes 112 initially in a direction indicated by arrow 950 (FIG. 9B) followed by a partial rotation in an opposite direction as indicated by arrow 960 (FIG. 9C). Oscillatory rotation of electrodes 112 could bring about rotational horizontal displacement of tissue folds 902 in the direction of the rotation as shown in FIGS. 9B and 9C deforming and massaging tissue below surface 400 down to a depth of approximately 1-10 mm.

As illustrated in FIGS. 9A-9C, tissue surface 400 orientation outside sink portion 320, i.e., outside circle 900, generally maintains its linear orientation throughout the oscillation cycle.

It will also be appreciated by persons skilled in the art that the present apparatus is not limited to what has been particularly shown and described hereinabove. Rather, the scope of the invention includes both combinations and sub-combinations mutatis mutandis of various features described hereinabove as well as modifications and variations thereof which would occur to a person skilled in the art upon reading the foregoing description and which are not in the prior art.

What we claim is:

1. In home-use cosmetic body contouring device, comprising:
a head including an oscillating carrier;
a convenient to hold handle integrally attached to the head; and
at least one curvilinear RF electrode.

2. The device according to claim 1, wherein the electrode is S-shaped.

3. The device according to claim 1, wherein the carrier is also operative to apply vibrating mechanical energy.

4. The device according to claim 1, also comprising at least one light energy emitting element selected from a group of light energy emitting elements including light illuminating diode (LED), laser, intense pulse light (IPL) and infrared (IR) energy emitter.

5. The device according to claim 1, also comprising a source of power, activating buttons, a controller and electronic circuitry.

6. The device according to claim 1, wherein the head also comprises a base integrally attached to the handle and having a centrally located bore defined by a floor and a ring-like wall having a rim and operative to rotatingly accommodate the carrier.

7. The device according to claim 1, wherein the head also comprises a motor attached to the oscillating carrier via a rotatable drive being at least one of rigid, telescopic or flexible.

8. The device according to claim 1, wherein the head also comprises at least one sensor selected from a group of sensors including temperature sensor, thermopile infrared sensor, pressure sensor, optical sensor, skin impedance sensor, movement sensor and positioning sensor.

9. The device according to claim 1, wherein the head also comprises a base having a centrally located bore defined by a floor and a ring-like wall having a rim; the carrier also comprises a surface; and
wherein the sensors are arranged along the rim or distributed on the surface of the carrier around and/or between RF electrodes.

10. The device according to claim 1, wherein the oscillating carrier is attached via a rotatable drive to an oscillating electrical motor located in at least one of the base and handle.

11. The device according to claim 1, wherein the carrier is depressed inside the bore so that the rim of the wall extends between 1-10 mm above surface of the carrier and defines a sink portion of the bore operative to accommodate a segment of tissue so that when the head is applied to surface the tissue, only a limited area of the surface of the tissue is urged into the sink portion and is allowed to come in contact with the surface of the carrier and the electrodes.

12. The device according to claim 6 wherein the ring-like wall rim absorbs most of the pressure applied by the tissue surface and the tissue urged into sink portion and being in contact with carrier surface does not apply excessive pressure to axis of a motor 304.

13. The device according to claim 1, wherein the oscillating carrier also mechanically exerts a massaging effect on and perpendicular to a limited portion of the tissue surface accommodated in a sink during oscillation of the carrier.

14. In home-use cosmetic body contouring device, comprising:
a convenient to hold handle;
a head having a discoid shape and a generally flat cross-section and including a base integrally attached to the handle and having a centrally located bore operative to rotatingly accommodate a rotatable carrier;
a rotatable carrier having a surface and operative to rotate up to 180 degrees or oscillate within the bore and having attached to its surface at least one of a rigid or semi-rigid curvilinear RF electrode;
a light emitting element; and
a sensor.

15. The device according to claim 14, wherein the surface of the rotatable carrier is curved.

16. The device according to claim 14, wherein the light emitting element is selected from a group of light energy emitting elements including a Light Emitting Diode (LED), laser source, intense pulse light (IPL), infrared (IR) energy emitter.

17. The device according to claim 14, wherein the sensor communicates with a controller in at least one of the handle or base.

18. The device according to claim 14, wherein the sensor is located on the surface of the carrier and/or on at least one of the electrodes.

19. The device according to claim 13, wherein the sensor is selected from a group of sensors including temperature sensor, thermopile infrared sensor, pressure sensor, optical sensor, skin impedance sensor, movement sensor and positioning sensor.

20. The device according to claims 1 and 14, wherein the cross-section of the head is at oval or semi-oval.

21. The device according to claim 5, wherein a source of power is at least one of a rechargeable battery and an external source of AC or DC power.

22. The device according to claim 10, wherein the motor is selected from a group of motors including brushless motors, ac or dc motors, stepper motors and piezoelectric motors.
